(19) United States
(12) Patent Application Publication
(10) Pub. No.: US 2016/0135768 A1
(43) Pub. Date: May 19, 2016

(54) SYSTEMS AND METHODS FOR DISPLAYING A PHYSIOLOGIC WAVEFORM

(71) Applicant: General Electric Company, Schenectady, NY (US)

(72) Inventors: Scott David Wollenweber, Waukesha, WI (US); Michael Joseph Cook, Oconomowoc, WI (US)

(21) Appl. No.: 14/542,744

(22) Filed: Nov. 17, 2014

(51) Int. Cl. A61B 6/00; A61B 5/00; A61B 5/113; A61B 6/04; A61B 6/03; A61B 5/0205

(52) U.S. Cl. CPC A61B 6/463; A61B 6/037; A61B 5/0205; A61B 5/113; A61B 6/0407; A61B 5/704; A61B 6/5288

(57) ABSTRACT

A method and system for displaying a physiologic waveform. The method and system acquire positron emission tomography (PET) coincidence event data of an object of interest. The method and system further select a subset of the PET coincidence event data corresponding to a time window and apply a multivariate data analysis technique to the subset of the PET coincidence event data. The method and system also generate a physiologic waveform based on the multivariate data analysis, and display the physiologic waveform on a display.

### BACKGROUND OF THE INVENTION

The subject matter disclosed herein relates generally to imaging systems, and more particularly to methods and systems for displaying a data-derived physiologic waveform from data acquired using a Positron Emission Tomography (PET) imaging system.

During operation of medical imaging system, such as PET imaging systems and/or multi-modality imaging systems (e.g., a PET/Computed Tomography (CT) imaging system, a PET/Magnetic Resonance (MR) imaging system), the image quality may be affected by the motion of the object being imaged (e.g., a patient). In particular, motion of the imaged object may create image artifacts during image acquisition, which degrades the image quality. Respiratory and cardiovascular motion is a common source of involuntary motion encountered in medical imaging systems. The respiratory motion may lead to errors during image review, such as when a physician is determining the size of a lesion, determining the location of the lesion, or quantifying the lesion.

While scanning patients, a clinician operating the medical imaging system may receive feedback on a display showing the current system performance which can be affected by the motion of the object being detected. For example, with data-driven gating a display of the average trigger rate may be shown. Optionally, the display may show real-time per second count rate as well as the remaining scan time. However, more detailed information, such as a physiologic waveform, may be needed by the clinician to determine whether user action is needed during the scanning

### BRIEF DESCRIPTION OF THE INVENTION

In an embodiment, a method is described for displaying a physiologic waveform. The method includes acquiring positron emission tomography (PET) coincidence event data of an object of interest. The method further includes selecting a subset of the PET coincidence event data corresponding to a time window and applying a multivariate data analysis technique to the subset of the PET coincidence event data. The method also includes generating a physiologic waveform based on the multivariate data analysis, and displaying the physiologic waveform on a display.

In an embodiment, a Positron Emission Tomography (PET) imaging system is provided. The PET imaging system includes a data acquisition controller configured to acquire PET coincidence event data from a detector ring assembly. The PET imaging system also includes a multivariate data analysis module (MDAM) communicatively coupled to the data acquisition controller. The MDAM is configured to select a subset of the PET coincidence event data corresponding to a time window and apply a multivariate data analysis technique to the subset of the PET coincidence event data. The MDAM is also configured to generate a physiologic waveform based on the multivariate data analysis. The PET imaging system also includes a display configured to display the physiologic waveform.

### BRIEF DESCRIPTION OF THE DRAWINGS

FIG. 1A is a flowchart of a method for displaying a physiologic waveform, in accordance with an embodiment.

FIG. 1B is a continuation of the flowchart of FIG. 1A.

FIG. 2 is a simplified block diagram of a positron emission tomography imaging system, in accordance with an embodiment.

FIG. 3 is an illustration of PET list data generated by the positron emission tomography imaging system of FIG. 2.

FIG. 4 is a graphical illustration of data plots from a subset of the positron emission tomography coincidence event data based upon spatial variation over time, aligned at a principal component axis, in accordance with an embodiment.

FIG. 5 is a graphical representation of a fast Fourier transform of the two dimensional graphical illustration of FIG. 4.

FIG. 6 is a graphical illustration of a physiologic waveform shown on a display of the positron emission tomography imaging system of FIG. 2.

FIG. 7 is an illustration of PET list data generated by the positron emission tomography imaging system of FIG. 2.

### DETAILED DESCRIPTION OF THE INVENTION

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. For example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated, such as by stating “only a single” element or step. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

“Systems,” “units,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

Generally, various embodiments provided herein describe using positron emission tomography (PET) list data acquired from a PET imaging system and/or multi-modality imaging system, such as a PET/CT imaging system, PET/MR imaging system, or the like, to generate a mask as well as derive principal components from the PET list data to generate a physiologic waveform. The physiologic waveform may correspond to respiratory movement, cardiovascular movement, or the like of the object of interest. The physiologic waveform may be generated based on a multivariate data analysis, such as a principal components analysis (PCA), when a predetermined count level or time duration of PET coincidence events are stored on the PET list data corresponding to scan locations of a patient. The physiologic waveform is displayed on a display after a fixed or variable time offset (e.g., back-off time 704 in FIG. 7) from the time of acquisition of the PET coincidence events corresponding to the physiologic waveform. The physiologic waveform may be updated to include subsequent subsets of the PET list data when additional PET coincidence events are acquired showing a near real-time derived physiologic waveform. When the acquisition of PET coincidence events ends, a final update of the physiologic waveform may be displayed.

A technical effect provided by various embodiments includes near real-time information displayed to the clinician corresponding to respiratory motion of a patient at the scan location. The near real-time physiologic motion waveform allows the clinician to instruct the patient if the scan protocol requires regular breathing. Another technical effect by various embodiments include allowing the clinician to adjust the acquisition time, for example, increasing the acquisition time at a scan location relative to a previous scan location.

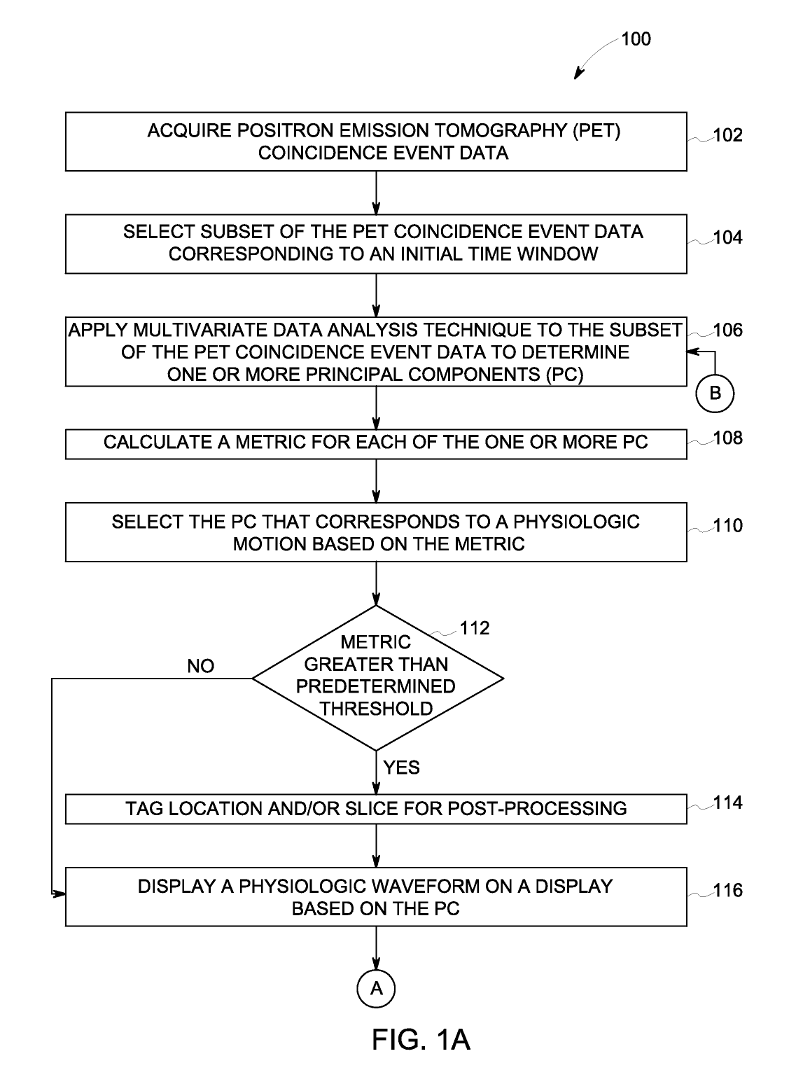

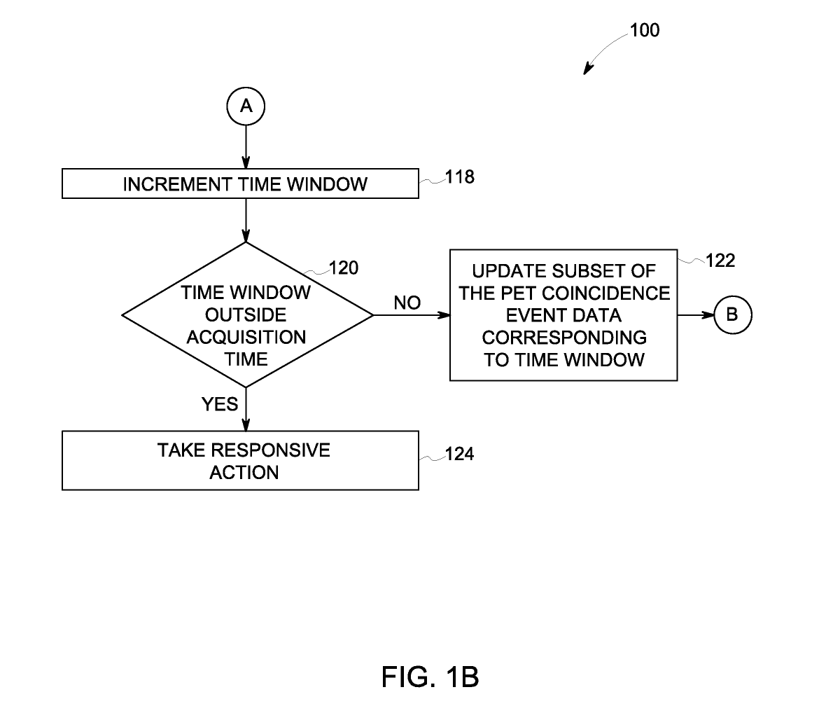

FIGS. 1a-b illustrates a flowchart of a method 100 for displaying a physiologic waveform. The method 100, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 100 may be used as one or more algorithms to direct hardware to perform one or more operations described herein. It should be noted, other methods may be used, in accordance with embodiments herein.

One or more methods may (i) acquire positron emission tomography (PET) coincidence event data of an object of interest; (ii) select a subset of the PET coincidence event data corresponding to a time window; (iii) apply a multivariate data analysis technique to the subset of the PET coincidence data; (iv) generate a physiologic waveform based on the multivariate data analysis; and (v) display the physiologic waveform on a display.

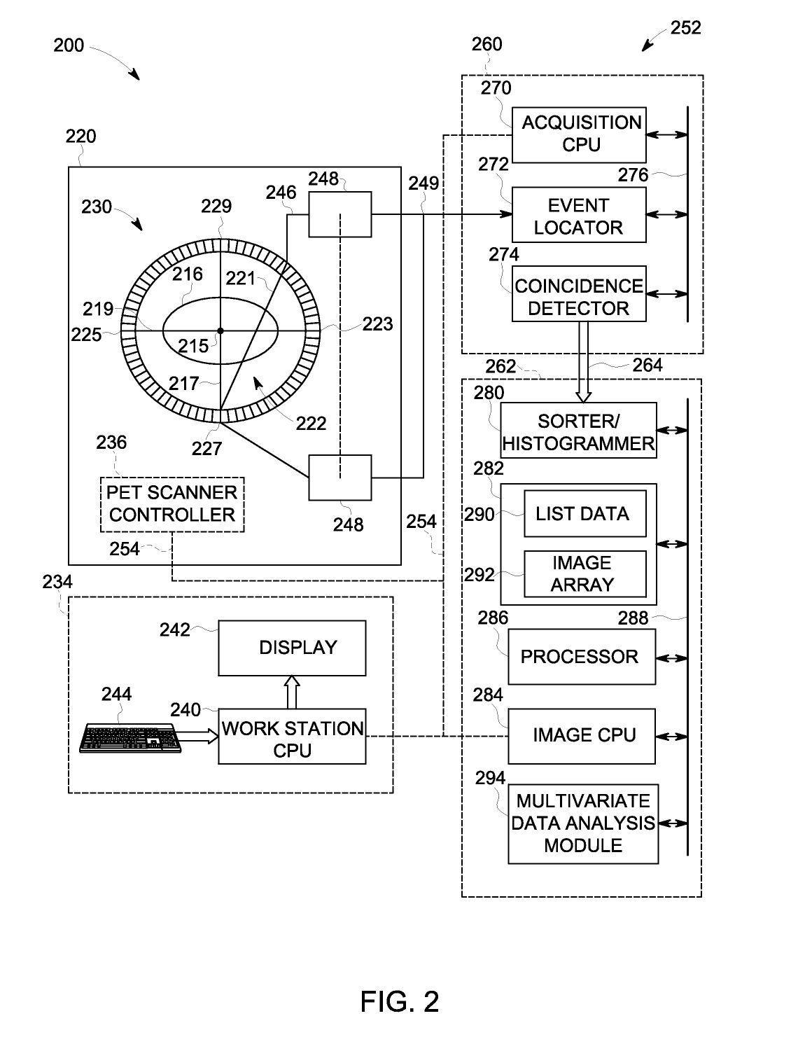

Beginning at 102, the method 100 acquires positron emission tomography (PET) coincidence event data. For example, the PET coincidence data may be acquired by a PET imaging system 200. FIG. 2 is a simplified block diagram of the PET imaging system 200, which may be used to acquire PET coincidence event data during a PET scan. The PET imaging system 200 includes a gantry 200, an operator workstation 234, and a data acquisition subsystem 252. In a PET scan, a patient 216 is initially injected with a radiotracer. The radiotracer comprises bio-chemical molecules that are tagged with a positron emitting radioisotope and can participate in certain physiological processes in the body of the patient 216. When positrons are emitted within the body, they combine with electrons in the neighboring tissues and annihilate creating annihilation events. The annihilation events usually result in pairs of gamma photons, with 511 keV of energy each, being released in opposite directions. The gamma photons are then detected by a detector ring assembly 230 within the gantry 220 that includes a plurality of detector elements (e.g., 223, 225, 227, 229). The detector elements may include a set of scintillator crystals arranged in a matrix that is disposed in front of a plurality of photosensors such as multiple photo multiplier tubes (PMTs) or other light sensors. When a photon impinges on the scintillator of a detector element, the photon produces a scintillation (e.g., light) in the scintillator. Each scintillator may be coupled to multiple photo multiplier tubes (PMTs) or other light sensors that convert the light produced from the scintillation into an electrical signal. In addition to the scintillator-PMT combination, pixilated solid-state direct conversion detectors (e.g., CZT) may also be used to generate electrical signals from the impact of the photons.

The detector ring assembly 230 includes a central opening 222, in which an object or patient, such as the patient 216 may be positioned, using, for example, a motorized table (not shown). The scanning and/or acquisition operation is controlled from an operator workstation 234 through a PET scanner controller 236. Typical PET scan conditions include data acquisition at several discrete table locations with overlap, referred to as ‘step-and-shoot’ mode. Optionally, during the PET scan may include the motorized table may traverse through the central opening 222 while acquiring PET coincidence event data, for example, a continuous table motion (CTM) acquisition. The motorized table during the CTM acquisition may be controlled by the PET scanner controller 236. During the CTM acquisition, the motorized table moves through the central opening 222 at a consistent or stable velocity (e.g., within a predetermine velocity threshold during the PET scan).

A communication link 254 may be hardwired between the PET scanner controller 236 and the workstation 234. Optionally, the communication link 254 may be a wireless communication link that enables information to be transmitted to or from the workstation 234 to the PET scanner controller 236 wirelessly. In at least one embodiment, the workstation 234 controls real-time operation of the PET imaging system 200. The workstation 234 may also be programmed to perform medical image diagnostic acquisition in reconstruction processes described herein.

The operator workstation 234 includes a work station central processing unit (CPU) 240, a display 242 and an input device 244. The CPU 240 connects to a communication link 254 and receives inputs (e.g., user commands) from the input device 244, which may be, for example, a keyboard, a mouse, a voice recognition system, a touch-screen panel, or the like. Through the input device 244 and associated control panel switches, the clinician can control the operation of the PET imaging system 200. Additionally or alternatively, the clinician may control the display 242 of the resulting image (e.g., image-enhancing functions), physiologic information (e.g., the scale of the physiologic waveform), the position of the patient 216, or the like, using programs executed by the CPU 240.

During operation of the PET imaging system, for example, one pair of photons from an annihilation event 215 within the patient 216 may be detected by two detectors 227 and 229. The pair of detectors 227 and 229 constitute a line of response (LOR) 217. Another pair of photons from the region of interest 215 may be detected along a second LOR 219 by detectors 223 and 225. When detected, each of the photons produce numerous scintillations inside its corresponding scintillators for each detector 223, 225, 227, 229, respectively. The scintillations may then be amplified and converted into electrical signals, such as an analog signal, by the corresponding photosensors of each detector 223, 225, 227, 229.

A set of acquisition circuits 248 may be provided within the gantry 220. The acquisition circuits 248 may receive the electronic signals from the photosensors through a communication link 246. The acquisition circuits 248 may include analog-to-digital converters to digitize the analog signals, processing electronics to quantify event signals and a time measurement unit to determine time of events relative to other events in the system 200. For example, this information indicates when the scintillation event took place and the position of the scintillator crystal that detected the event. The digital signals are transmitted from the acquisition circuits 248 through a communication link 249, for example, a cable, to an event locator circuit 272 in the data acquisition subsystem 252.

The data acquisition subsystem 252 includes a data acquisition controller 260 and an image reconstruction controller 262. The data acquisition controller 260 includes the event locator circuit 272, an acquisition CPU 270 and a coincidence detector 274. The data acquisition controller 260 periodically samples the signals produced by the acquisition circuits 248. The acquisition CPU 270 controls communications on a back-plane bus 276 and on the communication link 254. The event locator circuit 272 processes the information regarding each valid event and provides a set of digital numbers or values indicative of the detected event. For example, this information indicates when the event took place and the position of the scintillator crystal that detected the event. An event data packet is communicated to the coincidence detector 274 through a communication link 276. The coincidence detector 274 receives the event data packets from the event locator circuit 272 and determines if any two of the detected events are in coincidence.

Coincidence may be determined by a number of factors. For example, coincidence may be determined based on the time markers in each event data packet being within a predetermined time period, for example, 12.5 nanoseconds, of each other. Additionally or alternatively, coincidence may be determined based on the LOR (e.g., 217, 219) formed between the detectors (e.g., 223 and 225, 227 and 229). For example, the LOR 217 formed by a straight line joining the two detectors 227 and 229 that detect the PET coincidence event should pass through a field of view in the PET imaging system 200. Events that cannot be paired may be discarded by the coincidence detector 274. PET coincidence event pairs are located and recorded as a PET coincidence event data packet that is communicated through a physical communication link 264 to a sorter/histogrammer circuit 280 in the image reconstruction controller 262.

The image reconstruction controller 262 includes the sorter/histogrammer circuit 280. During operation, the sorter/histogrammer circuit 280 generates a PET list data 290 or a histogram, which may be stored on the memory 282. The term “histogrammer” generally refers to the components of the scanner, e.g., processor and memory, which carry out the function of creating the PET list data 290. The PET list data 290 includes a large number of cells, where each cell includes data associated with the PET coincidence events. The PET coincidence events may be stored in the form of a sinogram based on corresponding LORs within the PET list data 290. For example, if a pair of PET gamma photons are detected by detectors 227 and 229, the LOR 217 may be established as a straight line linking the two detectors 227 and 229. This LOR 217 may be identified as two dimensional (2-D) coordinates (r, θ, Δt), wherein r is the radial distance of the LOR from the center axis of the detector ring assembly 230, θ is the trans-axial angle between the LOR 217 and the X-axis, and Δt is the change in time of the detection of the photons between the two detectors 227 and 229 of the LOR 217. The detected PET coincidence events may be recorded in the PET list data 290. As the PET scanner 200 continues to acquire PET coincidence events along various LORs (e.g., 217, 219, 221), these events may be binned and accumulated in corresponding cells of the PET list data 290. The result is a 2-D sinogram λ(r, θ, Δt), each of which holds an event count for a specific LOR. In another example, for a three dimensional (3D) sinogram, an LOR 217, 219 may be defined by four coordinates (r, θ, z, Δt), wherein the third coordinate z is the distance of the LOR from a center detector along a Z-axis.

Additionally, the communication bus 288 is linked to the communication link 252 through the image CPU 284. The image CPU 284 controls communication through the communication bus 288. The array processor 286 is also connected to the communication bus 288. The array processor 286 receives the PET list data 290 as an input and reconstructs images in the form of image arrays 292. Resulting image arrays 292 are then stored in a memory module 282. The images stored in the image array 292 are communicated by the image CPU 284 to the operator workstation 246.

It should be noted that in at least one embodiment the PET coincidence event data may be acquired during a PET pre-scan, pauses during a PET scan, or PET coincidence event data that may not be used to reconstruct images on the image array 292. For example, the PET coincidence event data may be acquired during a pre-screening at target beds (e.g., couch positions) or over the diaphragm of the patient 216 to facilitate a pre-scan patient coaching on the breathing (e.g., respiratory movement) by the clinician. In another example, the PET coincidence event data may be acquired during a pre-scan for tuning of the PET scan prescription and/or planned motion mitigation.

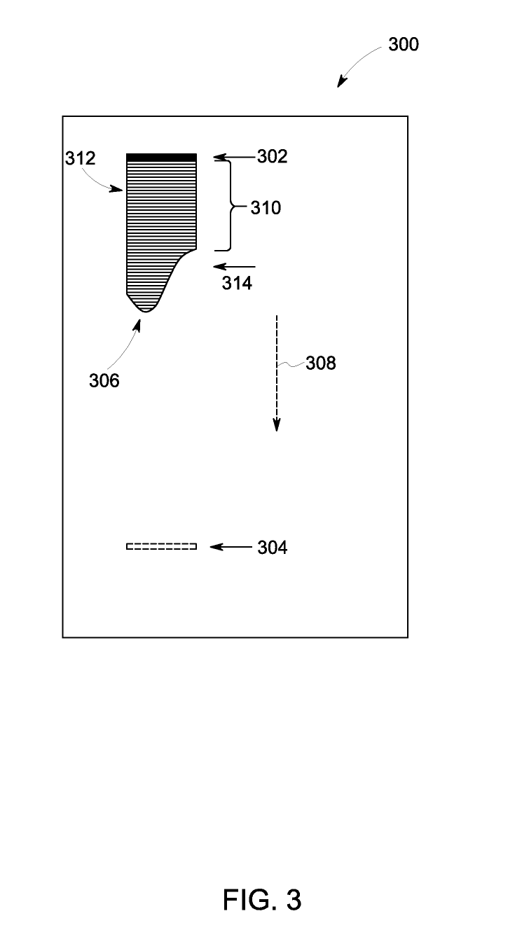

Returning to FIG. 1A, at 104, the method 100 selects a subset 312 of the acquired PET coincidence event data corresponding to an initial time window 310. FIG. 3 is an illustration 300 of the PET list data 306 being generated by the PET imaging system 200. The PET list data 306 is organized sequentially in time from a start time 302 of the PET scan to an end time 304 of the PET scan. For example, the PET coincidence event data acquired at the start or beginning of the PET scan is at a start time 302 of the PET list data 306. During the PET scan, additional PET coincidence event data is added to the PET list data 306 by the PET imaging system 200 in the direction of an arrow 308 until the end time 304 is reached. The amount of time from the start time 302 to the end time 304 corresponds to an acquisition time for the PET scan. An acquisition marker 314 may indicate a real time or position during the PET scan of new photons being detected by the detectors (e.g., the detectors 223, 225, 227, 229) relative to the start time 302 and the end time 304. As the scan progresses and additional cells are added to the PET list data 306, the acquisition marker 314 moves in the direction of the arrow 308 towards the end time 304.

The subset 312 corresponds to cells of the PET list data 306 or, specifically, PET coincidence event data that is within the initial time window 310. The initial time window 310 may be determined by a multivariate data analysis module (MDAM) 294 based on inputs received from the clinician through the operator workstation 234. Additionally or alternatively, the initial time window 310 may correspond to a sample size of the PET coincidence event data from the start time 302 to be used by the MDAM 294. Optionally, the initial time window 310 may be based on a selection by the clinician. In at least one embodiment, the initial time window 310 may be based on a predetermined amount of time during the PET scan, such as, ten to thirty seconds, and/or dependent on the target physiological signal (e.g., respiratory, cardiac, or the like). Additionally or alternatively, the initial time window 310 may be based on a minimum number of samples, a count level or a count level per sample of the PET list data 306 within the subset 312. Optionally, the initial time window 310 may be based on the type of multivariate data analysis to be used by the PET imaging system 200 (e.g., amount of data needed to determine a covariance matrix for principal component analysis (PCA)). It should be noted that in at least one embodiment, the subset 312 may include PET list data 306 up to a back-off time (e.g., the back-off time 704 in FIG. 7) from the acquisition marker 314 (e.g., real time). For example, the initial time window 310 may not include PET coincidence event data acquired at the acquisition marker 314 and/or during the back-off time.

At 106, the method 100 applies a multivariate data analysis technique to the subset of the PET coincidence data 312 to determine one or more principal components (PC). For example, the multivariate data analysis technique may be performed by the MDAM 294. Such multivariate data analysis techniques may include, for example, PCA, Independent Component Analysis (ICA), regularized PCA (rPCA), or the like. It should be noted, that although other analysis techniques may be utilized, many of which use PCA as an initial step.

The PCA technique is generally known and widely available in the art. In summary, the PCA technique finds the dominant eigenvectors from a covariance matrix based on the sorted subset of PET coincidence data 312. The covariance matrix is based on an average sinogram calculated from the subset of the PET coincidence data 312 and measures a deviation of each dimension from the mean with respect to each other. For example, the subset of the PET coincidence data 312 corresponds to a set of 3-D sinograms defined by (r, θ, z, Δt). The MDAD 294 may calculate a mean sinogram from the set of 3D sinograms based on a mean for each component (e.g., r, θ, z). The MDAD 294 may use the mean sinogram to determine the covariance matrix by subtracting the mean sinogram from each of the 3D sinograms from the set of 3D sinograms, and then summing the result. From the covariance matrix, the MDAD 294 may calculate eigenvectors and eigenvalues. The eigenvectors with the largest eigenvalue may correspond to the largest variations or dominant PC of the set of 3D sinograms over time. The MDAD 294 calculates one or more PC, for example, by multiplying the eigenvectors with the sorted PET coincidence data.

Optionally, the MDAD 294 may output or select one or more PC corresponding to the eigenvector having the largest magnitude eigenvalue. For example, the MDAD 294 may calculate three PC based on the 3D sinogram. The MDAD 294 may select the PC having the highest eigenvalue relative to the remaining PC. Additionally or alternatively, the MDAD 294 may display each PC on the display 242, and select one or more PC based on selections received from the input device 244.

Optionally, at 108, the method 100 calculates a metric for each of the one or more PC. Generally, the metric is intended to be a measure of signal strength related to the amount and/or type of physiologic motion. The MDAD 294 may calculate a metric for each of the one or more PC to determine which PC corresponds and/or closely relates to physiologic motion (e.g., a high metric relative to the other PC). The metric, such as a physiologic signal strength (PSS) metric, can be based on a frequency analysis corresponding to a ratio between a peak frequency 504 within a physiologically meaningful frequency window 508 to a mean above the physiologic frequency window 508.

For example, the physiologic frequency window 508 may be a frequency range that generally corresponds to periodic movement (e.g., physiologic motion) of the patient 216, such as, respiratory movement, cardiovascular movement, or the like. The PSS metric may be used to determine the amount of noise not related to the physiologic motion (e.g., frequencies outside the physiologic frequency window 508). For example, a PC with a high PSS metric may correspond to the PC including variances caused by physiologic movement. In another example, a PC with a low PSS metric may correspond to variances caused by noise, non-physiologic movement (e.g., shifting of the patient 216 during the PET scan), or the like.

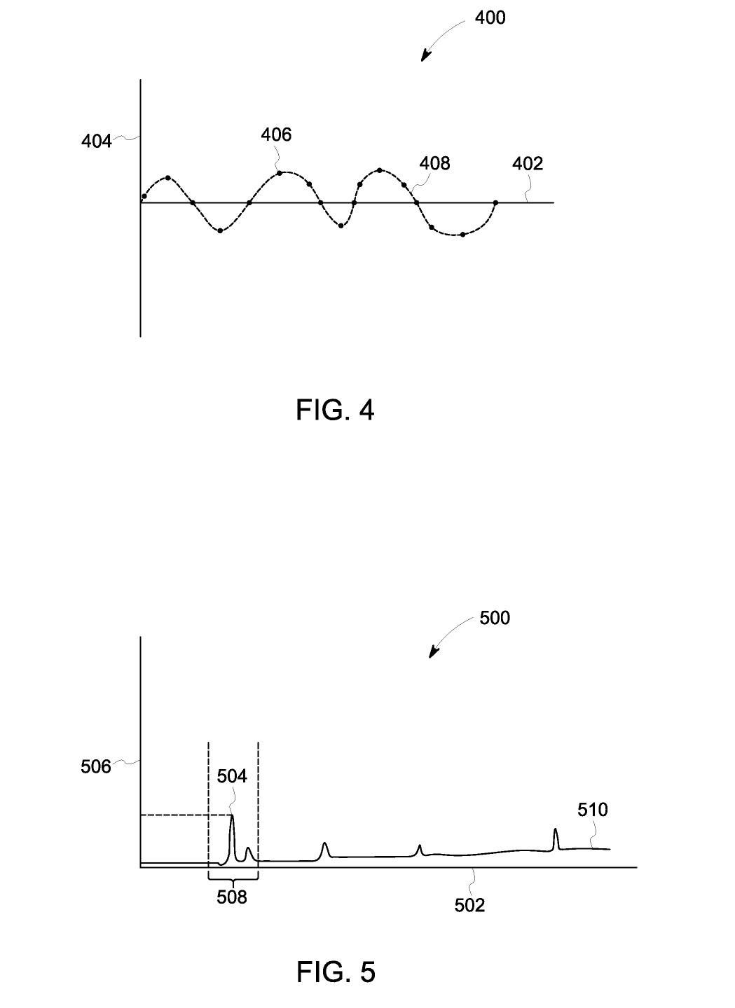

FIG. 4 is a graphical illustration 400 of data plots 406 based from the subset of the PET coincidence events aligned at a PC axis 404 calculated by the MDAD 294 and plotted 406 over time 402. The PC axis 404 is based on one of the PC calculated at 106. The data plots 406 represent a variance of each PET coincidence event within the subset of the PET coincidence events relative to the mean of the subset. The data plots 406 may be connected to represent a component waveform 408 or potential physiologic waveform. Optionally, based on the component waveform 408 and/or data plots 406, the MDAD 294 may perform a frequency analysis to determine a metric.

FIG. 5 is a graphical representation 500 of a Fast Fourier Transform 510 of the component waveform 408 from FIG. 4, for example, calculated by the MDAD 294. The horizontal axis 502 represents frequency, and a vertical axis 506 represents magnitude. The physiologic frequency window 508 may represent a frequency range typical for respiratory motion, for example, between 0.1 and 0.4 hertz (2.5 s-10 s period). Optionally, the physiologic frequency window 508 may be selected by the clinician through the operator workstation 234. Additionally or alternatively, there may be more than one physiologic frequency window 508 corresponding to different physiologic movements, for example, a first physiologic frequency window representing a frequency range for cardiovascular motion and a second physiologic frequency window representing a frequency range for respiratory motion. For multiple physiologic frequency windows, for example, the MDAD 294 may calculate multiple metrics (e.g., a respiratory signal strength metric, a cardiovascular signal strength metric, or the like) based on each physiologic frequency window corresponding to a physiologic movement.

The MDAD 294 may compare the magnitudes of frequencies within the physiologic frequency window 508 to determine the peak frequency 504 within the physiologic frequency window 508. The MDAD 294 may calculate a metric by dividing the magnitude of the peak frequency 504 by a mean of the frequency magnitudes corresponding to frequencies greater than or above the physiologic frequency window 508.

Optionally at 110, the method 100 selects the PC that corresponds to a physiologic motion based on a metric. For example, the MDAD 294 may compare the metrics (e.g., the PSS metrics, the respiratory signal strength metrics, the cardiovascular signal strength metrics) calculated for a plurality of PC, and select the PC with the greatest or highest metric relative to the other PC.

At 112, the method 100 determines whether the metric is greater than a predetermined threshold. If the metric is greater than the predetermined threshold, at 114, the method 100 may optionally tag a location and/or slide for post-processing. For example, the MDAD 294 may compare the metric of the selected PC with a predetermined threshold stored on the memory 282. The predetermined threshold may be based on a signal strength related to the amount of—physiologic motion for motion mitigation techniques performed by the image CPU 285. For example, image slices corresponding to the tagged location and/or slide may be used by the image CPU 285 to determine rejection of data during a non-quiescent portion of the breathing cycle.

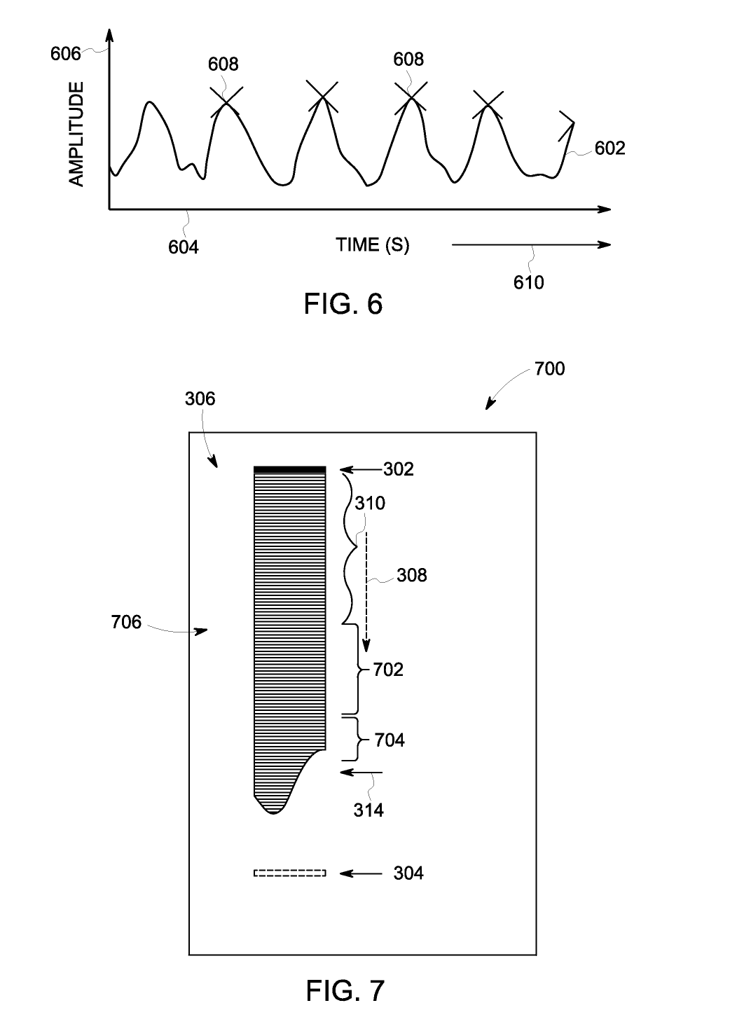

At 116, the method 100 displays a physiologic waveform 602 on the display 242 based on the PC. FIG. 6 is a graphical illustration of the physiologic waveform 602 that may be shown on the display 242 of the PET imaging system 200. A vertical axis 606 represents an amount of movement (e.g., amplitude) and a horizontal axis 604 represents time. The MDAD 294 may derive the physiologic waveform 602 from the component waveform 408 corresponding to physiologic motion of the patient 216 determined at 110 and communicated to the display 242 via the communication link 254. For example, the MDAD 294 may filter the component waveform 408 with a mid-pass filter based on the peak frequency 504 to remove frequencies (e.g., noise) not corresponding to the physiologic motion. Additionally or alternatively, the MDAD 294 may display the R metric of the PC concurrently or simultaneously with the physiologic waveform 602. Optionally, the scale of the physiologic waveform 602, width of the physiologic waveform 602, position of the physiologic waveform 602 within the display 242, color of the physiologic waveform 602, or the like, may be adjusted by the clinician via the input device 244. Additionally or alternatively, the operator workstation 234 may allow the clinician to adjust the zoom, add cursors (e.g., for 4-D gating during the PET scan), or the like. Optionally, the physiologic waveform 602 may include a gate trigger marker 608 indicating trigger positions of the physiologic waveform 602 for data-driven respiratory gating.

At 118 (FIG. 1B), the method 100 increments the time window 702. For example, after a predetermined wait period, the MDAD 294 may increase the size of the time window 702 to include PET coincidence event data acquired by the detectors (e.g., 223, 225, 227, 229) subsequent to the subset 312 of PET coincidence event data and/or outside the initial time window 310. For example, the increased size of the time window corresponds to a larger subset of the PET coincidence data relative to the initial time window 310. The additional PET coincidence data, included in the larger subset, was acquired after the PET coincidence data in the subset 312. Additionally or alternatively, the MDAD 294 may reposition the time window 702 to include PET coincidence event data not included within the subset 312 and acquired after the subset 312. The repositioning of the time window may correlate to a change in patient positioning (e.g., couch position) relative to the field of view of the PET detector 200.

The predetermined wait period may be based on an amount of time needed by the PET imaging system 200 to acquire enough PET coincidence data to fill (e.g., based on the number of cells or count level of the PET list data 306) an updated subset 706 corresponding to the incremented time window 702. Additionally or alternatively, the predetermined wait period may be based on a selection by the clinician received by the MDAD 294 via the operator workstation 234.

FIG. 7 is an illustration 700 of the PET list data 306 generated by the PET imaging system 200. The PET list data 306 in FIG. 7 includes additional PET coincidence event data acquired during the PET scan compared to the PET list data 306 shown in FIG. 3. For example, the PET list data 306 includes additional cells or count levels in the direction of the arrow 308 than the PET list data 306 shown in FIG. 3. The incremented time window 702 is shown adjacent (e.g., in relation to the PET list data 306) to the initial time window 310. It should be noted that in at least one embodiment the time window 702 is not adjacent to the initial time window 310. For example, a portion of the PET list data 306 may be interposed between and not within (e.g., not included in the subsets 312, 706) the incremented time window 702 and the initial time window 310.

Optionally, the updated subset 706 included within the incremented time window 702 may include PET list data 306 up to a back-off time 704 from the acquisition marker 314 (e.g., real time). The back-off time 704 may be based on the performance of the PET imaging system 200. For example, the back-off time 704 may be based on the amount of time between the detection of a photon by the detectors (e.g., the detectors 223, 225, 227, 229) of a PET coincidence event to when the PET coincidence event data is stored within the PET list data 290 on the memory 282.

At 120, the method 100 determines whether the time window 702 is outside the acquisition time. If the time window is within the acquisition time, at 122, the method 100 updates the subset (e.g., the updated subset 706) of the PET coincidence event data corresponding to the time window 702. For example, the MDAD 294 increments the initial time window 310 to form the time window 702. The updated subset 706 of the PET list data 306 within and/or corresponding to the time window 702 is before the end time 304 of the PET scan (e.g., when the PET imaging system 200 stops acquiring PT coincidence event data, or a significant change in bed positioning relative to the PET detector axial field-of-view). Since the time window 702 is before the end time 304, the MDAD 294 updates the subset 310 used at 106 for the multivariate data analysis technique with the new subset 706 that is within the time window 702. Optionally, if the MDAD 294 determines that the time window 702 is outside the acquisition time period, the MDAD 294 may adjust the size of the time window 702 to fit within the acquisition time (e.g., before the end time 304).

Based on the new subset 706, an alternative PC may be selected when the MDAD 294 applies the multivariable data analysis at step 106, relative to the subset 310. For example, based on the new subset 706 a new mean sinogram may be calculated by the MDAD 294 resulting in a new covariance matrix, which may result in different dominant PC selected by the MDAD 294, different values of the metrics for each PC, or the like. Optionally, the MDAD 294 may use the same PC based on the subset 310 from the initial time window 310 selected at 110.

It should be noted in at least one embodiment the MDAD 294 may include previous subsets (e.g., 310) within the updated subset 706. For example, the MDAD 294 may apply the multivariable data analysis to a subset that includes the subset 310 and the updates subset 706.

Additionally or alternatively, as additional physiologic waveforms are determined from the updated subsets (e.g., the updated subset 706). Optionally, the display 242 may scroll or update the physiologic waveform 602 dynamically as the physiologic waveform 602 is calculate by the MDAD 294, for example, during CTM acquisitions, to display a near real-time data derived physiologic waveform as new subsets are selected by the MDAD 294. The scrolling of the physiologic waveform 602 on the display 242 allows the clinician to continuously view a historical trend of the physiologic waveform 602 during the PET scan allowing the clinician to observe abrupt changes in physiologic movement, increase/decrease in the rate of physiologic movement, or the like. Optionally, the display may show a predetermined time range of the physiologic waveform 602 as the physiologic waveform 602 is scrolled or updated. For example, the horizontal axis 604 representing time maybe scrolled or shifted in the direction of an arrow 610, while additional calculations of the physiologic waveform 602 is added. Optionally, the MDAD 294 may only display segments of the physiologic waveform 602 on the display 242 corresponding to a single subset (e.g., the subset 310, the updated subset 706).

In at least one embodiment, the display 264 may also display an average metric, the metric for a single subset (e.g., the subset 310, the updated subset 706), or statistical information for the physiologic waveform 602 (e.g., standard deviation for the different amplitudes, standard deviation for peak frequencies for each subset). The average metric may be calculated by the MDAD 294 corresponding to a mean metric of the PC for the subsets 310, 706. Optionally, the above statistical information may be displayed in response to a selection by the clinician from the input device 244.

At 124, the method 100 takes responsive action. For example, a responsive action may be the display 264 showing a physiologic waveform corresponding to physiologic motion during the entire PET scan and/or more than one selected subset (e.g., the subsets 310, 706, all selected subsets) during the PET scan. In another example, the responsive action may be the MDAD 294 generating a summary

Optionally, during the PET scan the clinician may adjust the acquisition time via the input device 244. For example, the metric shown on the display 264 may be decreasing over the course of the PET scan, which may indicate an increase in noise within the PET list data 290. Based on an increasing or decreasing metric, the input device 244 may receive instruction to increase/decrease the PET scan or the acquisition time. For example, the extended acquisition time may correspond to increasing the stop time 304 by moving the stop time 304 in the direction of the arrow 308. In another example, for the CTM acquisition the adjustment in the acquisition time may also include changing the speed of the motorized table (e.g., increasing the velocity of the motorized table going into/out of the central opening 222, decreasing the velocity of the motorize table going into/out of the central opening 222). Optionally, the input device 244 may receive multiple increments of time increased for the acquisition time. Additionally or alternatively, the amount of the extended acquisition time may be based on a 4D gating corresponding to a position of the motorized table and/or patient 216 relative to the central opening 222, gantry 220, detector ring assembly 230, or the like.

Optionally, the physiologic waveform 602 may only be determined when the subset (e.g., 312, 706) or time window (e.g., 310, 702) of the PET list data (e.g., 306) corresponds to a select range or predetermined regions of interest of the patient 216 located approximate to the physiologic movement, a source of the physiologic movement, susceptible to the physiologic movement, or the like. The regions of interest and/or the select range, for example, may be selected by the clinician through the operator workstation 234. Optionally, the regions of interest and/or the select range may be based on computer tomography (CT) preliminary scans or a pre-PET CT scan. The region of interest and/or the select range may correspond to an organ (e.g., the lungs, the heart, kidneys, bladder, liver), an anatomical range (e.g., between the bladder and the top of the lungs), a portion of the body (e.g., chest, head, torso). Additionally or alternatively, the region of interest and/or the selected range may be based on a selection on the type of the physiologic waveform 602 to be displayed.

For example, before the PET scan using CTM acquisition, the input device 244 may receive an input to monitor respiratory movement. Based on the selection of respiratory movement, the operator controller 234 may instruct the MDAD 294 to calculate physiologic waveforms 602 corresponding to respiratory movement. The MDAD 294 may only select subsets of the PET list data (e.g., 306) from PET coincidence data located proximate to the lungs and/or an anatomical range of the patient 216 between the bladder and the top of the lungs.

It should be noted that the particular arrangement of components (e.g., the number, types, placement, or the like) of the illustrated embodiments may be modified in various alternate embodiments. For example, in various embodiments, different numbers of a given module or unit may be employed, a different type or types of a given module or unit may be employed, a number of modules or units (or aspects thereof) may be combined, a given module or unit may be divided into plural modules (or sub-modules) or units (or sub-units), one or more aspects of one or more modules may be shared between modules, a given module or unit may be added, or a given module or unit may be omitted.

As used herein, a structure, limitation, or element that is “configured to” perform a task or operation may be particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a processing unit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optic drive, and the like. The storage device may also be other similar means for loading computer programs or other instructions into the computer or processor.

As used herein, the term “computer,” “controller,” “system,” and “module” may each include any processor-based or microprocessor-based system including systems using microcontrollers, reduced instruction set computers (RISC), application specific integrated circuits (ASICs), logic circuits, GPUs, FPGAs, and any other circuit or processor capable of executing the functions described herein. The above examples are exemplary only, and are thus not intended to limit in any way the definition and/or meaning of the term “module” or “computer.”

The computer, module, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, module, or processor as a processing machine to perform specific operations such as the methods and processes of the various embodiments described and/or illustrated herein. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, and also to enable a person having ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

The foregoing description of certain embodiments of the present inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general purpose signal processor, microcontroller, random access memory, hard disk, or the like). Similarly, the programs may be stand alone programs, may be incorporated as subroutines in an operating system, may be functions in an installed software package, or the like. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “comprises,” “including,” “includes,” “having,” or “has” an element or a plurality of elements having a particular property may include additional such elements not having that property.

What is claimed is:

1. A method for displaying a physiologic waveform, said method comprising:
acquiring positron emission tomography (PET) coincidence event data of an object of interest;
selecting a subset of the PET coincidence event data corresponding to a time window;
applying a multivariate data analysis technique to the subset of the PET coincidence event data;
generating a physiologic waveform based on the multivariate data analysis; and
displaying the physiologic waveform on a display.

2. The method of claim 1, wherein the multivariate data analysis technique further comprises applying a principal component analysis (PCA) to the subset of the PET coincidence event data.

3. The method of claim 1, further comprising selecting a new subset of the PET coincidence event data corresponding to an adjusted time window;
repeat the applying operation based on the new subset of the PET coincidence event data;
generating an updated physiologic waveform based on the multivariate data analysis from the new subset and includes the physiologic waveform;
displaying the updated physiologic waveform.

4. The method of claim 1, wherein the physiologic waveform is based on a principal component derived from the multivariate data analysis technique.

5. The method of claim 1, further comprising calculating a metric for each PC, wherein the multivariate data analysis technique generates a plurality of PC for the subset of the PET coincidence event data.

6. The method of claim 5, further comprising selecting one of the PC based on the metric value relative to the other PC metric values, wherein the physiologic waveform is based on the selected PC.

7. The method of claim 1, wherein the physiologic waveform corresponds to at least one of respiratory movement or cardiovascular movement of the object of interest.

8. The method of claim 1, further comprising continually adjusting the time window to include a larger subset of the PET coincidence data relative to the subset; and
repeating the applying, generating and displaying operations based on each adjusted time window.

9. The method of claim 1, wherein the time window is based on an anatomical range of the patient corresponding to the physiologic movement.

10. The method of claim 1, further comprising adjusting an acquisition time based on the physiologic waveform.

11. The method of claim 1, wherein the acquiring operation is based on a continuous table motion (CTM) acquisition during a PET scan.

12. The method of claim 11, wherein a velocity of a motorized table during the CTM is adjusted based on the physiologic waveform.

13. A positron emission tomography (PET) imaging system comprising:
a data acquisition controller configured to acquire PET coincidence event data from a detector ring assembly;
a multivariate data analysis module (MDAM) communicatively coupled to the data acquisition controller, the MDAM configured to
select a subset of the PET coincidence event data corresponding to a time window;
apply a multivariate data analysis technique to the subset of the PET coincidence event data;
generate a physiologic waveform based on the multivariate data analysis; and
a display configured to display the physiologic waveform.

14. The PET imaging system of claim 13, wherein the multivariate data analysis technique of the MDAM applies a principal component analysis (PCA) to the subset of the PET coincidence event data.

15. The PET imaging system of claim 13, wherein the physiologic waveform is based on a principal component derived from the multivariate data analysis technique.

16. The PET imaging system of claim 13, wherein the MDAM further calculates a metric for each PC, wherein the multivariate data analysis technique generates a plurality of PC for the subset of the PET coincidence event data.

17. The PET imaging system of claim 16, wherein the MDAM further selects one of the PC based on the metric value relative to the other PC metric values, wherein the physiologic waveform is based on the selected PC.

18. The PET imaging system of claim 13, wherein the physiologic waveform corresponds to at least one of respiratory movement or cardiovascular movement of the object of interest.

19. The PET imaging system of claim 13, wherein the MDAM continually adjusts the time window to include a larger subset of the PET coincidence data relative to the subset; and
repeats the applying, generating and displaying operations based on each adjusted time window.

20. The PET imaging system of claim 13, wherein the timing window is based on an anatomical range of the patient corresponding to the physiologic movement.
